(19) United States
(12) Patent Application Publication
(10) Pub. No.: US 2023/0075150 A1
(43) Pub. Date: Mar. 9, 2023

(54) Urostomy Drain Tube Device

(71) Applicant: Brian Smith, St. Catharines (CA)

(72) Inventor: Brian Smith, St. Catharines (CA)

(21) Appl. No.: 17/551,571

(22) Filed: Dec. 15, 2021

Related U.S. Application Data
(60) Provisional application No. 63/241,688, filed on Sep. 8, 2021.

(51) Int. Cl. A61F 5/44; A61F 5/445

(52) U.S. Cl. CPC A61F 5/4407; A61F 5/445

(57) ABSTRACT

The present invention relates generally to a urostomy drain tube device primarily comprised of a first end that is further comprised of a receiver, and a second end that is further comprised of an outlet and a cap, wherein both ends are connected by tubing. The receiver of the first end preferably attaches to the existing nipple or opening on the bottom of a conventional urostomy pouch, and the button allows a user to control the flow of urine from the pouch into the receiver. Thus, once the button is depressed, urine can enter the receiver and travel through the tubing and out the outlet into a toilet or urinal.

### CROSS-REFERENCE TO RELATED APPLICATION

The present application claims priority to, and the benefit of, U.S. Provisional Application No. 63/241,688, which was filed on Sep. 8, 2021 and is incorporated herein by reference in its entirety.

### FIELD OF THE INVENTION

The present invention relates generally to the field of urostomy pouches. More specifically, the present invention relates to a urostomy drain tube device primarily comprised of a first end that is further comprised of a receiver, and a second end that is further comprised of an outlet and a cap, wherein both ends are connected by tubing. The receiver of the first end preferably attaches to the existing nipple or opening on the bottom of any urostomy pouch known in the art, and the button allows a user to control the flow of urine from the pouch into the receiver. Accordingly, the present disclosure makes specific reference thereto. Nonetheless, it is to be appreciated that aspects of the present invention are also equally applicable to other like applications, devices and methods of manufacture.

### BACKGROUND

Urostomy pouches/bags are used to collect urine after a urostomy (bladder surgery). After a urostomy, a patient's urine will go through their stoma into a urostomy pouch. Once the pouch fills with urine, it must be emptied. However, urostomy pouches known in the art only have a small release at the bottom of the pouch. As a result, a user with a urostomy pouch must often drain their pouch into a sink to avoid having to reach into a toilet to fully access the release while draining a urostomy pouch. This process is extremely undesirable, as a sink must be thoroughly cleaned each time after a urostomy pouch has been drained. In addition, some users may be embarrassed to drain a urostomy pouch into the sink of a public restroom, in the event that they must drain the pouch while in public.

Therefore, there exists a long-felt need in the art for an improved urostomy pouch device. There also exists a long-felt need in the art for a urostomy bag drain tube device that can be attached to a urostomy pouch and that does not require a user to drain the pouch into a sink. In addition, there exists a long-felt need in the art for a urostomy bag drain tube device that allows a user to discretely drain a urostomy bag while in public.

The subject matter disclosed and claimed herein, in one embodiment thereof, comprises a urostomy drain tube device. The device is comprised of a first end that is further comprised of a receiver, and a second end that is further comprised of an outlet and a cap, wherein both ends are connected by tubing. The receiver of the first end preferably attaches to the existing nipple or opening on the bottom of a conventional urostomy pouch, and the button allows a user to control the flow of urine from the pouch into the receiver. Thus, when the button is not depressed, urine cannot enter the receiver; and when the button is depressed, urine can enter the receiver and travel through the receiver, through the tubing and out the outlet into a toilet or urinal.

In this manner, the urostomy drain tube device of the present invention accomplishes all of the forgoing objectives and provides an improved means to drain a urostomy pouch. The device does not require that a user drain a urostomy pouch into a sink, and instead allows the user to discretely empty the pouch into a toilet or urinal. As a result, the device provides a more private, less embarrassing and convenient method of draining a urostomy pouch. Therefore, the device greatly improves the quality of life of users who live with urostomy pouches.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a urostomy drain tube device primarily comprised of a first end that is further comprised of a receiver, and a second end that is further comprised of an outlet and a cap, wherein both ends are connected by tubing. The receiver of the first end preferably attaches to the existing nipple or opening on the bottom of any urostomy pouch or urostomy bag known in the art. The receiver is also preferably hollow, and has a first continuous opening that slides over the existing nipple of the pouch in a removable or fixedly-secured fashion. In one embodiment of the device, the receiver is a clip-like member that can be clipped to the nipple of a pouch. The receiver is also comprised of at least one button that allows a user to control the flow of urine from the pouch into the receiver, such that when the button is not depressed urine cannot enter the receiver. However, when the button is depressed, urine can enter the receiver. As a result, the button allows urine to remain in the pouch while the device is attached to the pouch and until a user is ready to drain the pouch. Furthermore, the receiver has a second continuous opening that further attaches into or around a piece of tubing that places the receiver in fluid communication with the second end.

The second end is further comprised of an outlet that has a first continuous opening and a second continuous opening. The outlet may be fixedly or removably-attached to the tubing and has a plurality of female threads that engage a plurality of male threads on the inside of a cap. Therefore, when a user desires to drain the pouch, the cap can be unscrewed from the outlet such that urine can flow from the pouch, through the receiver, through the tubing, into the outlet via the first opening and out of the outlet via the second opening.

The invention may also comprise a method of using the device. To use the device, a user first attaches the receiver to the nipple or opening of a urostomy pouch/bag. Then, a user unscrews the cap from the outlet and positions the tubing and outlet near or into a toilet or urinal. Once adequately positioned, a user can then depress the button to allow urine from the pouch to flow through the receiver, through the tubing and out through the outlet into the urinal or toilet. After all urine has been drained from the pouch, a user can then re-screw the cap onto the outlet and remove the device from the pouch. However, a user may also choose to keep the device attached to the pouch at all times, instead of removing the device from the pouch after all urine has been drained. Using the method, a user can discretely use the device to empty urine from a urostomy pouch while standing at a urinal. Additionally and/or alternatively, a user can discretely use the device to empty urine from a urostomy pouch while standing or sitting on a conventional toilet within a bathroom or bathroom stall.

Accordingly, the urostomy drain tube device of the present invention is particularly advantageous as it provides a more private, less embarrassing and more convenient method of draining a urostomy pouch/bag. In addition, the device does not require a user to drain a urostomy pouch into a sink, but instead offers the user the option of discretely emptying the pouch into a toilet or urinal. As a result, the device greatly improves the quality of life of users who live with urostomy pouches.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the urostomy drain tube device of the present invention, wherein the urostomy drain tube device is attached to a urostomy pouch in accordance with the disclosed architecture;

FIG. 2 illustrates an enhanced perspective view of one potential embodiment of the urostomy drain tube device of the present invention in accordance with the disclosed architecture;

FIG. 3 illustrates a flowchart of one method of using one potential embodiment of the urostomy drain tube device of the present invention in accordance with the disclosed architecture; and

FIG. 4 illustrates a perspective view of one potential embodiment of the urostomy drain tube device of the present invention in use at a urinal in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for an improved urostomy pouch device. There also exists a long-felt need in the art for a urostomy pouch drain tube device that can be attached to a urostomy pouch, wherein the device does not require a user to drain the pouch into a sink. In addition, there exists a long-felt need in the art for a urostomy bag drain tube device that allows a user to discretely drain a urostomy pouch while in public, and to avoid any embarrassment and inconvenience associated with draining a urostomy pouch into a sink in a public or private setting.

The present invention, in one exemplary embodiment, is comprised of a urostomy drain tube device. The device is primarily comprised of a first end that is further comprised of a receiver, and a second end that is further comprised of an outlet and a cap, wherein both ends are connected by tubing. The receiver of the first end preferably attaches to the existing nipple or opening on the bottom of any urostomy pouch or urostomy bag known in the art. Further, the receiver is also preferably hollow and has a first continuous opening that slides over the existing nipple of the pouch in a removable or fixedly-secured fashion. In one embodiment of the device, the receiver is a clip-like member that can be clipped to the nipple of a pouch, and is preferably comprised of at least one button that allows a user to control the flow of urine from the pouch into the receiver. Thus, when the button is not depressed, urine cannot enter the receiver; and when the button is depressed, urine can enter the receiver. Therefore, the button allows urine to remain in the pouch while the device is attached to the pouch, until a user is ready to drain the pouch.

Furthermore, the receiver has a second continuous opening that further attaches into or around a piece of tubing that places the receiver in fluid communication with the second end. The second end is further comprised of an outlet that has a first continuous opening and a second continuous opening. The outlet may be fixedly or removably-attached to the tubing and has a plurality of female threads that engage a plurality of male threads on the inside of a cap. Therefore, when a user desires to drain the pouch, the cap can be unscrewed from the outlet such that urine can flow from the pouch, through the receiver, through the tubing, into the outlet via the first opening and out of the outlet via the second opening.

The invention may also comprise a method of using the device. To use the device, a user first attaches the receiver to the nipple or opening of a urostomy pouch. Then, a user unscrews the cap from the outlet, and positions the tubing and outlet near or into a toilet or urinal. Once adequately positioned, a user can then depress the button to allow urine from the pouch to flow through the receiver, through the tubing and out through the outlet into the urinal or toilet. After all urine has been drained from the pouch, a user can then re-screw the cap onto the outlet and remove the device from the pouch. However, a user may also choose to keep the device attached to the pouch instead of removing the device from the pouch after all urine has been drained. Using this method, a user can discretely use the device to empty urine from a urostomy pouch while standing at a urinal. Additionally and/or alternatively, a user can discretely use the device to empty urine from a urostomy pouch while standing or seated on a conventional toilet within a bathroom or bathroom stall.

Accordingly, the urostomy drain tube device of the present invention is particularly advantageous as it provides a more private, less embarrassing and convenient method of draining a urostomy pouch. In addition, the device does not require a to user drain a urostomy pouch into a sink, but instead offers the user the option of discretely emptying the pouch into a toilet or urinal. As a result, the device greatly improves the quality of life of users who live with urostomy pouches/urostomy bags.

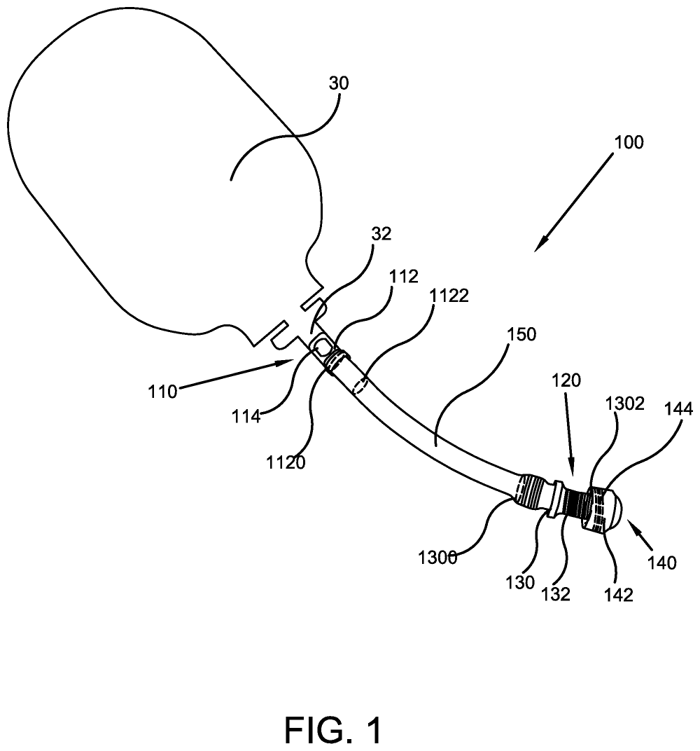

Referring initially to the drawings, FIG. 1 illustrates a perspective view of one embodiment of the urostomy drain tube device 100 of the present invention, which is attached to a urostomy pouch/bag 30 (shown as prior art). The device 100 is primarily comprised of a first end 110 that is further comprised of a receiver 112, and a second end 120 that is further comprised of an outlet 130 and a cap 140, wherein both ends 110, 120 are connected by a semi-flexible or flexible plastic-based tubing 150. The receiver 112, outlet 130 and cap 140 are preferably comprised of a plastic, such as but not limited to: acrylic, polycarbonate, polyethylene, thermoplastic, acrylonitrile butadiene styrene, low density polyethylene, medium density polyethylene, high density polyethylene, polyethylene terephthalate, polyvinyl chloride, polystyrene, polylactic acid, acetal, nylon, fiberglass, etc. In a differing embodiment, the receiver 112, outlet 130 and cap 140 may be manufactured of a durable metal such as, but not limited to, stainless steel or aluminum, etc., or any other suitable material as is known in the art. In addition, all components of the device 100 may have an anti-bacterial/anti-microbial coating to ensure the device 100 remains sterile.

The receiver 112 of the first end 110 preferably attaches to the existing nipple 32 or opening on the bottom of a conventional urostomy pouch/bag 30. However, in one embodiment, the device 100 may include an integrated urostomy pouch/bag 30 as part of the invention. The receiver 112 is preferably hollow, and has a first continuous opening 1120 that slides over the existing nipple 32 of the pouch 30 in a removable or fixedly-secured fashion. In one embodiment of the device 100, the receiver 112 is a clip-like member that can be clipped to the nipple 32 of a pouch 30. The receiver 112 is also further comprised of at least one button 114. The button 114 allows a user to control the flow of urine from the pouch 30 into the receiver 112. Thus, when the button 114 is not depressed, urine cannot enter the receiver 112; and when the button 114 is depressed, urine can enter the receiver. In this manner, the button 114 allows urine to remain in the pouch 30 while the device 100 is attached to the pouch 30, until a user is ready to drain the pouch 30. Further, the receiver 112 has a second continuous opening 1122 that attaches into or around the piece of plastic or vinyl tubing 150, which places the receiver 112 in fluid communication with the second end 120.

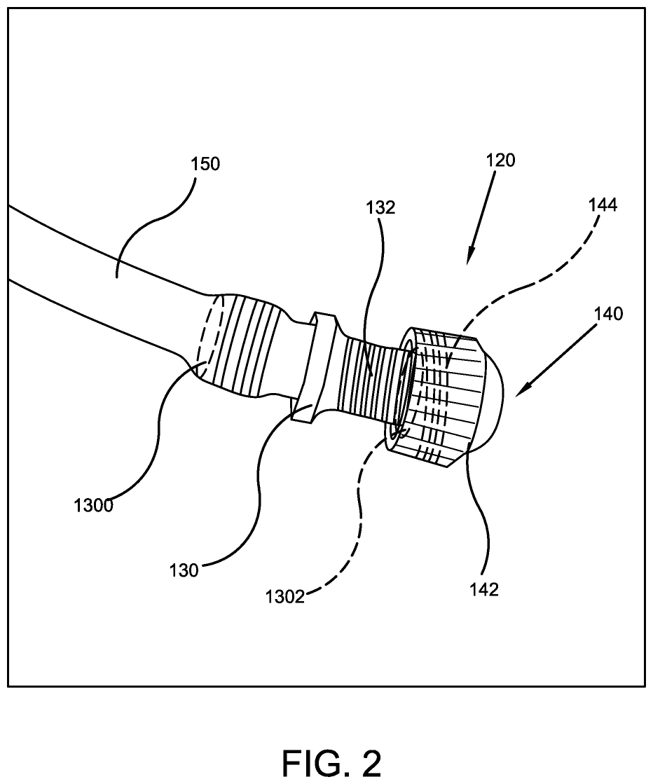

FIG. 2 illustrates an enhanced perspective view of one embodiment of the urostomy drain tube device 100 of the present invention. The second end 120 is further comprised of an outlet 130 that has a first continuous opening 1300 and a second continuous opening 1302, wherein the outlet 130 may be fixedly or removably-secured to the tubing 150. Further, the outlet 130 has a plurality of female threads 132 that engage a plurality of male threads 144 on the inside of a cap 140. When a user desires to drain the pouch 30, the cap 140 can be unscrewed from the outlet 130, such that urine can flow from the pouch 30, through the receiver 112, through the tubing 150, into the outlet 130 via the first opening 1300 and out of the outlet 130 via the second opening 1302. To aid a user in screwing/unscrewing the cap 140, the body 142 of the cap 140 may further be textured to promote a better grip and ease of use.

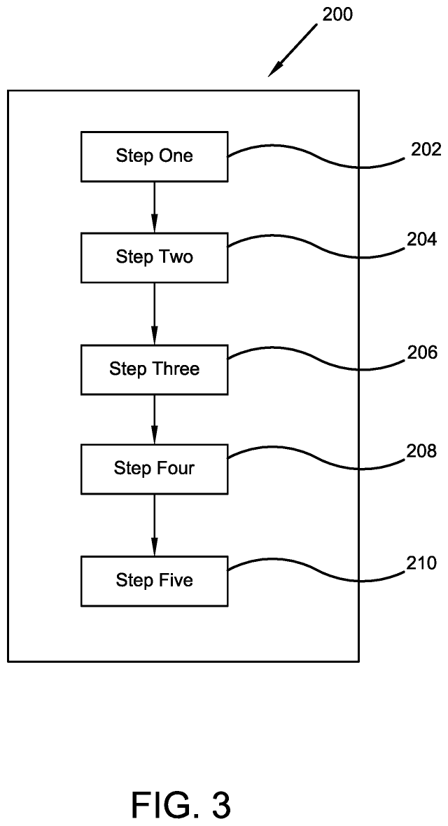

FIG. 3 illustrates a flowchart of one method 200 of using the urostomy drain tube device 100 of the present invention. To use the device 100, at 202, a user first attaches the receiver 112 to the nipple 32 or opening of a urostomy pouch. Then at 204, a user unscrews the cap 140 from the outlet 130. Next at 206, a user positions the tubing 150 and outlet 130 near or into a toilet or urinal 20. Once adequately positioned, at 208 a user can then depress the button 114 to allow urine from the pouch 30 to flow through the receiver 112, through the tubing 150, and out through the outlet 130 into the urinal 20 or toilet. After all urine has been drained from the pouch 30, at 210 a user can then re-screw the cap 140 onto the outlet 130, and remove the device 100 from the pouch 30. However, it should be appreciated that a user may also keep the device 100 attached to the pouch 30, instead of removing the device 100 from the pouch 30.

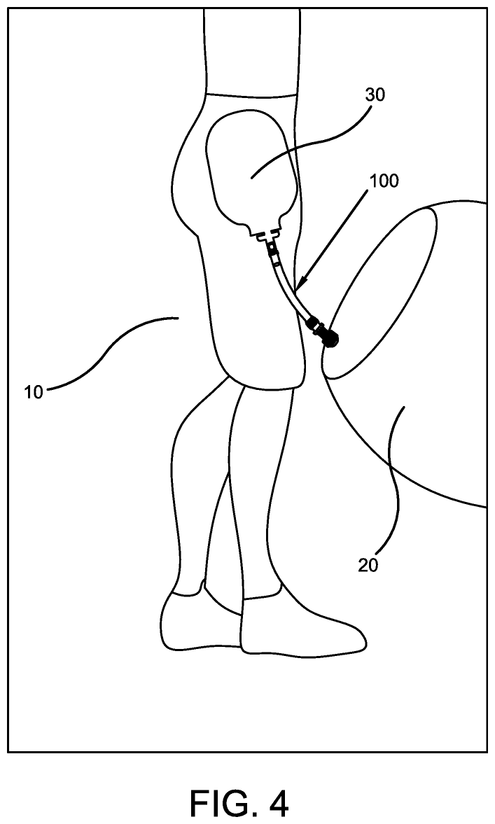

FIG. 4 illustrates a perspective view of one embodiment of the urostomy drain tube device 100 of the present invention in use in a urinal 20 (shown as prior art). Using the method 200, a user 10 can discretely use the device 100 to empty urine from a urostomy pouch 30 while standing at a urinal 20. Additionally and/or alternatively, a user 10 can discretely use the device 100 to empty urine from a urostomy pouch 30 while standing or seated on a conventional toilet within a bathroom or bathroom stall. In this manner, a user 10 can avoid the undesirable and unsanitary practice of emptying urine from a pouch 30 into a sink. Further, the device 100 eliminates any embarrassment that may otherwise be experienced by a user 10, when visually emptying a pouch 30 without the device 100.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “urostomy drain tube device” and “device” are interchangeable and refer to the urostomy drain tube device 100 of the present invention. Further, as used herein “urostomy bag” and “urostomy pouch” are interchangeable and refer to a device used to hold the urine of patients who have had a urostomy or other similar medical procedure.

Notwithstanding the forgoing, the urostomy drain tube device 100 of the present invention and its various components can be of any suitable size and configuration as is known in the art without affecting the overall concept of the invention, provided that they accomplish the above-stated objectives. One of ordinary skill in the art will appreciate that the size, configuration and material of the urostomy drain tube device 100 as shown in FIGS. 1-4 is for illustrative purposes only, and that many other sizes and shapes of the urostomy drain tube device 100 are well within the scope of the present disclosure. Although the dimensions of the urostomy drain tube device 100 are important design parameters for user convenience, the urostomy drain tube device 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A urostomy drain tube device comprising:
a first end comprised of a receiver;
a second end comprised of an outlet and a cap;
wherein the first end and the second end are connected by a flexible tubing;
wherein the first end secures to an opening on a bottom of a conventional urostomy pouch;
wherein the receiver comprises a button that controls flow of urine from the urostomy pouch through the receiver; and
wherein the outlet and the cap are secured to the flexible tubing such that when the cap is removed, urine can flow from the urostomy pouch, through the receiver, through the flexible tubing, and exit the outlet.

2. The urostomy drain tube device of claim 1, wherein the outlet is fixedly secured to the flexible tubing.

3. The urostomy drain tube device of claim 1, wherein the outlet is removably secured to the flexible tubing.

4. The urostomy drain tube device of claim 1, wherein the outlet comprises a plurality of female threads which engage a plurality of male threads on inside of the cap, to secure the cap to the outlet.

5. The urostomy drain tube device of claim 4, wherein the cap can be removed from the outlet during use and can be replaced onto the outlet when not in use.

6. A urostomy drain tube device that can be attached to a urostomy pouch to allow a user to drain the urostomy pouch into a toilet or a urinal, the urostomy drain tube device comprising:
a first end comprised of a hollow receiver;
a second end comprised of an outlet and a cap;
wherein the first end and the second end are connected by a flexible tubing, such that the flexible tubing connects to the receiver and to the outlet;
wherein the first end secures to an opening on a bottom of a conventional urostomy pouch;
wherein the hollow receiver comprises a button that controls flow of urine from the urostomy pouch through the hollow receiver, a first continuous opening, and a second continuous opening;
wherein the outlet comprises a first continuous opening, a second continuous opening, and a plurality of female threads;
wherein the cap secures to the outlet via the plurality of female threads; and
wherein the outlet and the cap are secured to the flexible tubing, such that when the cap is removed, urine can flow from the urostomy pouch, through the hollow receiver, through the flexible tubing, and out the outlet.

7. The urostomy drain tube device of claim 6, wherein the first continuous opening of the receiver slides over the opening of the conventional urostomy pouch in a removable or fixedly secured fashion.

8. The urostomy drain tube device of claim 7, wherein the button allows a user to control the flow of urine from the conventional urostomy pouch into the hollow receiver, such that when the button is not depressed urine cannot enter the hollow receiver and when the button is depressed urine can enter the hollow receiver.

9. The urostomy drain tube device of claim 6, wherein the plurality of female threads of the outlet engages a plurality of male threads on an inside surface of the cap.

10. The urostomy drain tube device of claim 6, wherein a user can discretely use the urostomy drain tube device to empty urine from the conventional urostomy pouch while standing at a urinal or sitting on a toilet.

11. The urostomy drain tube device of claim 6, wherein the urostomy drain tube device has an anti-bacterial or anti-microbial coating to ensure the urostomy drain tube device remains sterile.

12. The urostomy drain tube device of claim 6, wherein the cap is textured to provide a better grip.

13. The urostomy drain tube device of claim 6, wherein the cap can be removed from the outlet during use and can be replaced onto the outlet when not in use.

14. The urostomy drain tube device of claim 6, wherein the hollow receiver is fixedly secured to the conventional urostomy pouch as one integrated component.

15. The urostomy drain tube device of claim 6, wherein the hollow receiver, the outlet, and the cap are comprised of an acrylic, a polycarbonate, a polyethylene, a thermoplastic, an acrylonitrile a butadiene styrene, a low density polyethylene, a medium density polyethylene, a high density polyethylene, a polyethylene terephthalate, a polyvinyl chloride, a polystyrene, a polylactic acid, an acetal, a nylon, or a fiberglass material.

16. The urostomy drain tube device of claim 6, wherein the hollow receiver, the outlet, and the cap are comprised of a stainless steel or an aluminum material.

17. The urostomy drain tube device of claim 6, wherein the hollow receiver is a clip-like member that can be clipped to the opening of the conventional urostomy pouch.

18. A method of using a urostomy drain tube device to drain urine from a conventional urostomy pouch into a toilet or a urinal, the method comprising the steps of:
attaching a receiver of the urostomy drain device to an opening of the conventional urostomy pouch;
unscrewing a cap from an outlet of the urostomy drain device;
positioning a tubing of the urostomy drain device and the outlet near or into a toilet or a urinal;
depressing a button to allow urine from the conventional urostomy pouch to flow through the receiver, through the tubing, and out through the outlet into the urinal or toilet;
re-screwing the cap onto the outlet; and
removing the urostomy drain device from the conventional urostomy pouch after all the urine has been drained from the conventional urostomy pouch.

19. The method of claim 18, wherein a user can re-screw the cap onto the outlet after draining urine and then keep the urostomy drain device secured to the conventional urostomy pouch.

20. The method of claim 18, wherein a user can discretely use the urostomy drain tube device to empty urine from a conventional urostomy pouch while sitting at a toilet or standing at a urinal.
